(19) United States
(12) Patent Application Publication
(10) Pub. No.: US 2011/0117122 A1
(43) Pub. Date: May 19, 2011

(54) COMPOSITION AND METHOD FOR AFFECTING CYTOKINES AND NF-kB

(76) Inventors: Tiffany Thomas, Scottsdale, AZ (US); Fazlul Sarkar, Plymouth, MI (US); Denis M. Callewaert, Metamora, MI (US); Andrew Dahl, Bloomfield Hills, MI (US); Enrique Martinez, Clinton Township, MI (US)

(21) Appl. No.: 12/947,684

(22) Filed: Nov. 16, 2010

Related U.S. Application Data
(60) Provisional application No. 61/261,639, filed on Nov. 16, 2009.

(51) Int. Cl. A61K 36/06

(52) U.S. Cl. 424/195.15

(57) ABSTRACT

The present invention discloses a composition and method for effecting various cytokines and NF-κB by administering an effective amount of a phyto-percolate composition to an individual. In various exemplary embodiments, the composition is claimed to be useful for the effective treatment of inflammation, cancer, and/or various infections including HIV by regulation of various interleukins, such as IL-10 and IL-2, and of transcription factors including NF-κB.

### CROSS-REFERENCE TO RELATED APPLICATION

This Application claims priority to and the benefit of U.S. Provisional Patent Application No. 61/261,639 filed Nov. 16, 2009, entitled “Composition and Method for Affecting Cytokines and NF-κB,” wherein such provisional application is hereby incorporated in its entirety.

### FIELD OF THE INVENTION

This invention generally relates to a composition and method for altering the production and/or function of proteins such as cytokines and transcription factors. More specifically, the present invention relates to a composition derived from the culture or co-culture of specific freshwater microorganisms, algae, moss, bacteria and/or fungi and a method of treating or preventing inflammation and/or diseases such as cancer and HIV by administering an effective amount of the composition.

### BACKGROUND

Cytokines are a broad class of proteins that are secreted by various cell types, including cells of the immune system. One function of cytokines is to carry various signals between cells and thus control activity among cells. Several factors can cause cells to secrete cytokines, including a cell's encounter with pathogens, which may cause disease. In certain instances, cells will secrete cytokines as a means of organizing a natural defense against the pathogen or disease.

There are numerous cytokines, many of which are commonly called interleukins (“IL”) produced by white blood cells. In turn, there are numerous different interleukins such as, for example, IL-2, IL-10, and IL-17A. Each of these different interleukins has specific functions and effects such as decreasing or increasing inflammation, stimulating the proliferation and function of various cell types and regulating the production of antibodies. For example, IL-2 and TNF-α contribute towards inflammation and may be considered as inflammatory proteins while IL-10 may be considered an anti-inflammatory protein that decreases inflammation. Therefore, the more IL-2 and TNF-a produced, the greater the inflammation. Conversely, the more IL-10 produced the less inflammation.

Interleukins have been determined to be involved in many processes, including, but not limited to, inflammation. For example, there is substantial evidence suggesting that IL-2 suppresses the production of immunoglobulins. In contrast, there is substantial evidence suggesting that IL-10 enhances immunoglobulin production.

Another cytokine is interferon-gamma or IFN-γ. IFN-γ is critical for innate and adaptive immunity against viral and intracellular bacterial defense functions and for tumor control. IFN-γ has been shown to alter the transcription of over thirty genes and to produce such affects as increasing Th2 cell activity, promoting NK cell activity, and affecting various other molecular signaling pathways.

Other cytokines include tumor necrosis factor alpha or TNF-α which is involved in the regulation of immune cells. Further, elevated production of TNF-α has been implicated as a contributing factor in a variety of human diseases, including cancer. Yet another cytokine is granulocyte-macrophage colony-stimulating factor or GM-CSF. GM-CSF is a white blood cell growth factor that is known to stimulate stem cells, and is part of the immune/inflammatory cascade.

A transcription factor known as “nuclear factor kappa beta” or NF-κB is an intracellular protein that functions as a regulator of gene transcription and plays an important role in various biological processes and pathology. NF-κB has been found to play an important role in regulating the immune system in response to infection and in several inflammatory pathways including the production of cyclooxygenase, nitric oxide synthase and other pro-inflammatory proteins. Inappropriate regulation of NF-κB has been linked to cancer, inflammatory and autoimmune diseases, septic shock, viral infection, and improper immune development and certain studies have linked NF-κB to processes involving synaptic plasticity and memory. The role of NF-κB and various cytokines is discussed in the article entitled Using Chemopreventive Agents to Enhance the Efficacy of Cancer Therapy by Sarkar, et al. and published by the American Association for Cancer Research on Apr. 1, 2006 which is herein incorporate by reference in its entirety. Further, various viruses, including the HIV virus have molecular binding sites for NF-κB thus indicating the NF-κB may be a critical component for activating the HIV virus from a latent state to an active state.

Therefore, the regulation of cytokines and/or NF-κB can be a critical process in providing treatment for various ailments. For example, since IL-10 has anti-inflammatory properties, increasing IL-10 in a patient suffering from a chronic inflammatory condition can be used to treat the inflammation. Alternatively, since NF-κB is a factor for activating the HIV virus from a latent state to an active state, reducing the amount of NF-κB could delay or prevent the HIV virus from being activated.

Currently, there are known compositions and methods for regulating cytokines and NF-κB. However, many of these known compositions and methods are irritating to cells or have a toxic effect on cells. Further, many known compositions and methods for regulating cytokines and NF-κB regulate many cytokines in the same manner, some of which may hinder the overall desired effect of the treatment. For example, there are known compositions and methods for treating inflammation that up-regulate anti-inflammatory cytokines such as IL-10, but these compositions also result in the up-regulation of IL-2, an inflammatory cytokine that reduces the effect of the IL-10.

Therefore, it would be advantageous to provide an improved composition and method of regulating anti-inflammatory cytokines and NF-κB and effected these on a cellular level. Moreover, providing a composition and method that could regulate selected cytokines and NF-κB to achieve a multitude of effects to treat various health problems would be desirable. One example of such specific regulation of multiple cytokines would be a composition that up-regulates IL-10 without up-regulating IL-2, or even while downregulating IL-2, thus increasing anti-inflammatory cytokines while reducing or maintaining the level of pro-inflammatory cytokines in order to reduce inflammation. It would also be desirable to provide a composition and method to affect various cytokines and NF-κB that is not an irritant, is non-toxic, is easy to manufacture and distribute, and is not expensive to produce.

### SUMMARY

As set forth in the detailed description and in accordance with various embodiments of the present invention, a composition and method for effecting cytokines and NF-κB is disclosed. According to one exemplary embodiment, the composition is derived from the culture or co-culture of specific freshwater microorganisms, algae, moss, bacteria and/or fungi of ATCC Deposit No. PTA-5863.

According to various exemplary embodiments of the present invention, a method of effecting cytokines and NF-κB to regulate immune response, reduce inflammation, provide antioxidant activity, modulate antibody production, treat or prevent cancerous tumor growth, and treat or prevent infections including HIV is disclosed. The composition is non-toxic and is capable of selectively up-regulating certain cytokines such as IL-10 while maintaining or reducing other cytokines such as IL-2 and/or TNF-α to achieve a desired result, such as reduced inflammation. In still yet other exemplary embodiments of the present invention, a method of affecting the DNA-binding activity of NF-κB and a method of reducing TNF-α-induced activation of NF-κB is disclosed. Further, according to various exemplary embodiments of the present invention, methods of inducing certain anti-inflammatory cytokines such as IL-10, particularly while not inducing other pro-inflammatory cytokines such as IL-2, TNF-α and IFN-γ is disclosed.

### BRIEF DESCRIPTION OF THE DRAWINGS

The subject matter of the invention is particularly pointed out and distinctly claimed in the concluding portion of the specification. Embodiments of the invention, however, may best be understood by reference to the following description taken in conjunction with the accompanying drawing figures.

FIGS. 1A-1D illustrate raw data from electrophoretic gel mobility shift assays according to various exemplary embodiments of the present invention;

FIG. 2 shows a bar graph illustrating the quantitative analysis of the results obtained in the experiment presented in FIGS. 1A-1D, thus illustrating the efficacy of the method effecting NF-κB according to various exemplary embodiments of the present invention;

FIG. 3 shows a bar graph illustrating the efficacy of the method on the production of the cytokine IL-2 according to various exemplary embodiments of the present invention;

FIG. 4 shows a bar graph illustrating the efficacy of the method on the production of the cytokine IL-10 according to various exemplary embodiments of the present invention;

FIG. 5 shows a bar graph illustrating the efficacy of the method on the production of the cytokine IL-17A according to various exemplary embodiments of the present invention;

FIG. 6 shows a bar graph illustrating the efficacy of the method on the production of the cytokine INF-γ according to various exemplary embodiments of the present invention;

FIG. 7 shows a bar graph illustrating the efficacy of the method on the production of the cytokine TNF-α according to various exemplary embodiments of the present invention; and

FIG. 8 shows a bar graph illustrating the efficacy of the method on the production of GM-CSF according to various exemplary embodiments of the present invention.

### DETAILED DESCRIPTION

The following description is of exemplary embodiments of the invention and is not intended to limit the scope or applicability of the invention in any way. Rather, the following description is intended to provide convenient illustrations for implementing various embodiments of the invention. Other configurations, compositions, amounts, and methods, and the like may be employed without departing from the scope of the present invention. As will become apparent, various other changes may be made to the methods described in these embodiments without departing from the spirit and scope of the invention.

According to various exemplary embodiments of the present invention, the present invention comprises administering a composition to affect various cytokines and NF-κB. The composition has been described in numerous commonly owned and co-pending patent applications including U.S. Pat. No. 7,807,622 entitled “Composition and Use of Phyto-Percolate For Treatment of Disease,” U.S. patent application Ser. No. 12/897,574 entitled “Composition and Use of Phyto-Percolate For Treatment of Disease,” U.S. patent application Ser. No. 11/587,266 entitled “Method of Preparation and Use of Fibrinolytic Enzymes in the Treatment of Disease,” U.S. Patent Application Ser. No. 61/306,591 entitled “Method of Lowering Cholesterol With PAZ Components,” and U.S. Patent Application Ser. No. 61/311,838 entitled “Agents and Mechanisms for Treating Hypercholesterol with PAZ Components,” all of which are herein incorporated by reference in their entirety. All foreign and PCT patent applications claiming priority to these U.S. applications are also incorporated herein by reference in their entirety.

The composition referred to herein as “phyto-percolate” is a non-toxic composition comprised generally of molecules produced by the culture or co-culture of specific microorganisms such as algae, moss, bacteria, and fungi. In one exemplary embodiment, a deposit of the culture used to create phyto-percolate has been placed in the American Type Culture Collection, of Manassas, Virginia as Deposit No. PTA-5863. This deposit is available to the public upon grant of a patent disclosing same. This deposit was made pursuant to 37 C.F.R. §1.808 and MPEP §2410.01 and therefore, access to the deposit will be available during pendency of this application making reference to the deposit to one determined by the Commissioner to be entitled thereto under 37 C.F.R. §1.14 and 35 U.S.C. §122 and with one exception, that all restrictions imposed by the depositor on the availability to the public of the deposited biological material be irrevocably removed upon granting of the patent.

In one exemplary embodiment, the composition described herein as “phyto-percolate” is created by the process set forth below. According to this embodiment, approximately one or more aliquots of the culture of the type currently on deposit as ATCC culture deposit number PTA-5863 are first obtained. In various embodiments where more than one aliquot is used, the aliquots may be combined in one larger composite culture vessel and maintained using the methods set forth below.

According to this exemplary embodiment, for each aliquot of culture obtained and cultured successfully from cryopreservation, the total volume is diluted using sterile deionized water to approximately 10 mL total volume (for example, 3 aliquots (−4.5 mL) are combined and diluted to 30 mL total volume). Further, a nutrient blend stock solution is prepared by mixing approximately 20 mg of dry active baker's yeast in approximately 1 mL warm sterile deionized water and then incubated for approximately 20 minutes at room temperature, yielding enough nutrient for approximately 1000 culture aliquots. Then, approximately 1 uL of the prepared nutrient blend is added to each diluted aliquot (for example, to 3 combined and diluted aliquots, add 3 uL prepared nutrient blend) and the mixture is then swirled gently.

The next step of producing phyto-percolate according to this exemplary embodiment comprises the step of incubating the culture sample with nutrient blend for approximately 1 week at room temperature in a sterilized culture vessel such as a round-bottom glass culture vessel with an ambient sterile-filtered air vent. In this exemplary embodiment, the mixture is swirled once half way through the week and maintained under approximately a 12:12 hour cycle of simulated daylight. After this week, approximately 1 uL freshly prepared nutrient blend is added to the culture vessel for approximately each diluted aliquot used and this new mixture is preferably swirled gently. The culture sample with nutrient blend is incubated for approximately one additional week at room temperature and preferably swirled once half way through the week and maintained under a 12:12 hour cycle of simulated daylight.

Continuing with this exemplary method of producing phyto-percolate, the liquid volume is slowly drawn off or harvested using a sterile tubing and siphon or peristaltic pump from approximately the top half of the culture vessel without disturbing the algal biomass growing in the bottom of the culture vessel, yielding approximately 5 mL per deposit aliquot used. The liquid may be reserved in a sterile glass storage container or another appropriate storage container, sterile-filtered and administered as desired. The liquid volume in the culture vessel should be replenished back to approximately its pre-harvested volume using sterile deionized room temperature water allowing the total final volume to be approximately 10 mL per deposit aliquot used. Approximately 1 uL of freshly prepared nutrient blend is then added to the culture vessel for approximately each aliquot used and then the mixture is swirled gently and allowed to incubate as described above in subsequent cycles as desired.

With continued reference to this exemplary embodiment, the culture sample and nutrient blend is incubated for approximately 1 week or more at room temperature while maintaining approximately a 12:12 hour cycle of stimulated daylight. While this culture is incubating with the nutrient blend, the previously harvested material is filtered through sterilizing membrane filters (or similar filters as those skilled in the art will recognize) with approximately a 0.2 um pore size to generate the final bioactive liquid, described herein as ‘composition’ or ‘phyto-percolate’. Any biomass captured in the filter may be destroyed or collected. Supplemental micronutrient or trace mineral blends specific to the needs of the culture may also be added to the culture during incubation or scale-up to preserve the integrity of the original culture biomass and support further growth.

Further, according to this exemplary manufacturing method, once sufficient biomass has been generated over time in the culture (approximately 8 to 12 weeks or more), the culture may be split into 2 equal cultures as needed in a scale-up process by the following exemplary steps. First, homogenize the culture gently to fully suspend the biomass. Second, transfer approximately half of the homogeneous culture into a new sterilized glass or other appropriate culture vessel. Third, replenish the liquid volume in each of the two culture vessels back to original culture volume using sterile deionized water at room temperature. Fourth, add approximately 1 uL of freshly prepared nutrient blend to each culture vessel and swirl gently. Fifth, incubate the cultures with nutrient blend for approximately 1 week at room temperature, preferably swirling once half way through the week and maintaining them under the approximate 12:12 hour cycle of simulated daylight. Sixth, add approximately an additional 1 uL freshly prepared nutrient blend to the culture vessel. Seventh, incubate the culture sample with nutrient blend for approximately an additional week at room temperature, preferably swirling once half way through the week. Finally, with respect to this scale-up process, it should be noted that multiple cultures can be combined in larger culture vessels and maintained using the same general culturing methods and nutrient-to-culture volume ratios.

With continued reference to this exemplary embodiment of producing phyto-percolate, the steps noted above should be proceeded as needed to generate a sufficient amount of phyto-percolate and its various derivatives. A sample of the phyto-percolate sold under the trademark PROALGAZME® may also be obtained from Health Enhancement Products, Inc. of Scottsdale, Ariz.

It should be noted that while specific examples have been given related to a method of producing a composition and quantities in the composition, that various modifications to the compositions and methods of producing the composition can be used and fall within the scope of the present invention. Further, it is contemplated and within the scope of the present invention that other culture methods, dilution volumes, growth media or nutrient blends, volumes or feeding frequencies, incubation times, culture vessels, harvesting or filtering methods, etc. may also be used to produce phyto-percolate and the exemplary method noted above is not intended to exclude other methods of producing phyto-percolate.

As used herein, the term phyto-percolate denotes the composition described above and derivatives thereof Phyto-percolate also denotes any composition that is made with the process described above or variations to that process that would be recognizable to one of ordinary skill in the art. Applicants reserve the right to more narrowly define the term “phyto-percolate” after this application has been filed.

Further, according to various exemplary embodiments of the present invention, the phyto-percolate is isolated into various fractions. Certain exemplary, non-limiting processes are described below.

According to one exemplary embodiment, the phyto-percolate is passaged in series through four chromatography columns with the dimensions of 2.7 cm×23 cm (approximately 100 mL of resin at full capacity each) at a flow rate of approximately ˜6 mL per minute using a pump such as a peristaltic pump. The rate is selected for optimal binding, and is also based on the flow rate of the slowest resin, (C18). The process is optimized to enable the fractionation of approximately 180 L of phyto-percolate. Other variations and modifications of these methods, including optimization of the process to accommodate other sample volumes, will be apparent to those of ordinary skill in the art. The chart below provides an exemplary flow chart for the separation and isolation of the constituents

Following passage of approximately 18 L through a resin, such as a DEAE resin, the column is replaced with a fresh column and the bound material from the prior approximate ˜18 L immediately eluted, filtered through a 0.2-micron filter and the eluates stored in sterile containers. Similarly and according to this exemplary embodiment, the anion and cation exchange resins are replaced after the passage of approximately ˜36 L of material through each. A single hydrophobic resin, (C18), is used for the entire process. All eluted fractions from the first three columns are immediately passaged through sterile filters and stored in sterile containers. Elution of the material bound to the C18 column requires the use of organic solvents, which are subsequently removed as detailed below. The material that does not bind to any of the four columns, having been depleted of the majority of the organic constituents, is labeled as the “flow-through” fraction and is collected into sterile containers for subsequent testing and use.

A detailed description of each step in the separation process is now described according to one exemplary embodiment of the present invention. First, the chromatography column resins are prepared by following the following process. DEAE Cellulose (weak anion exchange resin widely used for isolation of proteins) is used in this exemplary process. Prior to use, DEAE cellulose must be pretreated with a strong base and acid solutions to strip off any contaminants that might interfere with the binding of proteins or contaminate the proteins thus isolated. Approximately twenty grams of DEAE-cellulose are rehydrated in approximately ˜300 mL of water (ultrapure water is used in this exemplary embodiment) and allowed to swell overnight or an equivalent time at room temperature in a 1 L flask. Water is decanted from the settled/packed resin and the resin is resuspended in an additional ˜300 mL of water such as ultrapure water. This resuspension and decanting procedure is repeated two more times through the course of approximately twenty-four hours. The washed resin is resuspended in ˜200 ml of 0.1 M NaOH/0.5 M NaCl then transferred to a 600 ml Buchner funnel according to this exemplary embodiment. The flask is then rinsed with an additional approximate 50 ml of 0.1 M NaOH/0.5 M NaCl and the material suspended in the rinse is also transferred to the funnel. The resin is allowed to sit in this solution for ˜10 minutes before allowing the solution to flow through by gravity. The resin is then rinsed with an additional ˜750 ml of 0.1M NaOH/0.5 M NaCl. This filtering procedure is then repeated using 0.5 M NaCl and again using 0.1 M HCl/0.5 M NaCl. The resin is initially rinsed with ˜2 L water such as ultrapure water followed by a further rinsing with ˜5 L of ultrapure water until the pH of the effluent is greater than five. The DEAE-cellulose slurry is then transferred to five columns (according to this exemplary embodiment, the five columns measure 2.7×23 cm) and allowed to settle. The packed columns have bed volumes of ˜100 ml and are stored at 4° C. until use in this exemplary embodiment.

Further, according to this exemplary embodiment, approximately 100 g of a dry resin such as BioRad AG 1-X8 Strong Base Anion Exchange Resin: Catalogue number 140-1441, received in chloride form, 100-200 dry mesh, 106-180 μm wet bead diameter, quaternary ammonium functionality, is used. To remove any unwanted oxidation contaminants, the resin is exhausted by first hydrating it with deionized water and then loading the beads into a glass column equipped with a glass filter at the bottom of each column. By passing approximately 500 mL of 1.0 M sodium chloride solution through the resin over a period of about three hours, the resin swells and releases any unwanted oxidation products. This process also converts the resin to a chloride (Cl—) form. After this salt treatment, the resin is rinsed with approximately two liters of deionized water to remove excess sodium chloride.

The anion exchange resin, now completely in the chloride (Cl—) form, is converted into the hydroxide (—OH) form by passing approximately 500 mL of 2.0 M sodium hydroxide solution through the column over a period of about two hours. The resin is subsequently rinsed with approximately 7.0 L of deionized water, overnight, using a gravity siphon drip as the effluent may be slightly off-color and have an ammonia-like odor. Following this step, the resin's effluent is clear, colorless, and odorless in this exemplary embodiment. The solution eluting from the column is pH neutral as measured with indicating strips. This anion exchange resin is now considered to be regenerated and ready for use.

Further, according to this exemplary embodiment, approximately 100 g of a dry resin such as DOWEX MONOSPHER® 88 Strong Acid Cation Exchange Resin: 400-700 μm bead diameter with sulfonate functionality available from the Dow Chemical company of Midland, Michigan is used. As for the anion exchange resin, unwanted oxidation contaminants are exhausted by first hydrating with deionized water and then loading the beads into a glass column equipped with a glass filter at the bottom of each column. Passage of approximately 500 mL of 1.0 M sodium chloride solution through the resin over a period of about three hours releases any unwanted oxidation products and removes any ions that may have been on the resin from production. The sodium chloride exhaustion causes the resin to convert completely to the sodium (Na+) form. After this salt treatment, the resin is rinsed with approximately 2.0 liters of deionized water to remove excess sodium chloride.

The cation exchange resin, now completely in the sodium (Na+) form, is converted to the acid (H+) form by passing approximately 500 mL of 2.0 M hydrochloric acid solution through the column over a period of about two hours. The resin is subsequently rinsed with ca. 3.0 L of deionized water, until the solution eluting from the column is pH neutral as measured with indicating strips. This cation exchange resin is now considered to be regenerated and ready for service.

Further, and in accordance with this exemplary embodiment, at the silica gel 90 C₁₈-Reversed phase (C-18), approximately 25 g of resin is resuspended in ultrapure water, packed into a column and washed with approximately 5 volumes of water prior to use.

Continuing on with the description of this exemplary embodiment, the following paragraphs provide a detailed timetable for the fractionation process. The phyto-percolate is pumped through columns set up in sequence such that the effluent from one column flows through to the next column, at a flow rate of approximately 6.9 ml/min. Additionally, collection vessels are cleaned and dried for flow-through collection. The saved flow-through is passaged through a 0.2 μm filter system and is stored at approximately 4-25° C.

After the first ˜18 L that passes through, the DEAE-cellulose column is removed and eluted with 250 ml 1M NaCl, pH 8.3. The eluate is filtered through a 0.2 g filter, labeled and stored at 4° C. Then, a fresh DEAE-cellulose column is placed into the fractionation system and the process resumed. After another —18 L are passaged, the DEAE-cellulose, anion exchange, and cation exchange columns are removed and each eluted with approximately 250 ml 1M NaCl, pH 8.3. The eluates are passaged through individual 0.2 μm filter systems, labeled and stored at approximately 4° C.

According to this exemplary embodiment, fresh DEAE-cellulose, anion exchange and cation exchange columns were placed into the fractionation system and the process resumed. After another ˜18 L, the DEAE-cellulose column is removed and eluted with 250 ml NaCl, pH 8.3. The eluate is passaged through a 0.2 μm filter system, labeled and stored at 4° C. Elution of material bound to the C18 column (from all material): The C-18 column is drained of excess water and purged with compressed nitrogen to remove residual water.

The column is then flushed with approximately 50 mL of acetone to remove the last traces of water and organics, followed by approximately 50 mL of ethyl acetate and finally approximately 50 mL of hexanes. The solution is then dried with excess anhydrous magnesium sulfate and filtered through glass wool or another similar material.

The solvent is then removed with a stream of nitrogen, and then reconstituted with approximately 5 mL of ethyl acetate and transferred to a glass vial of known mass. The solvent is removed with nitrogen and the final mass is taken.

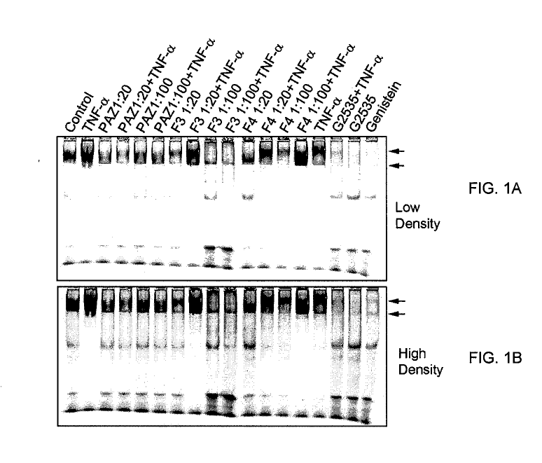

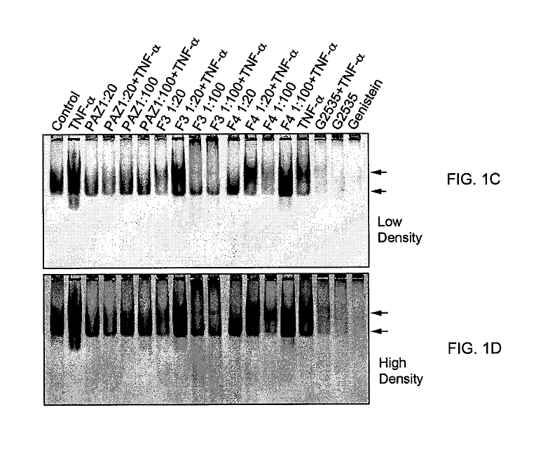

Further, the DEAE-cellulose, anion exchange, and cation exchange columns were each eluted with approximately 250 ml 1M NaCl, pH 8.3. The eluates were passaged through individual 0.2 μm filter systems, labeled and stored at approximately 4° C. One mL of eluate from the cation exchange column (labeled as Fraction 3 or “ F3” in FIGS. 1-8 and described in the present invention) is the eluate captured from the cation exchange columns after the phyto-percolate has passed through the first three columns using the methods described above and is approximately 160 fold concentrated compared to the unseparated phyto-percolate introduced into the separation process (i.e. for every 160 mL of phyto-percolate introduced into the process, one mL of eluate was isolated in PF3).

Fraction 4 as labeled as F4 in FIGS. 1-8 and described in the present invention is the flow-through captured at the end of the fractionation series after the phyto-percolate has passed through all 4 columns using the methods described above.

In the experiments for which results are presented in FIGS. 1-8, the dilutions provided are those of the completed, unseparated phyto-percolate composition or of the specific fractions identified. For example, since the total volume of flow-through isolated in F4 is identical to that of the unfractionated phyto-percolate, the relative concentration(s) of all constituents in F4 was identical to that of the unseparated phyto-percolate, whereas the relative concentration of constituents in a 1:20 dilution of the F3 fraction eluted from the strong cation exchange resin is approximately 8 fold concentrated relative to unseparated phyto-percolate (since one mL of F3 is obtained for every 160 mL of phyto-percolate, a 1:20 dilution equates to the constituents therein being approximately 8 fold concentrated relative to unseparated phyto-percolate).

According to this exemplary embodiment, phyto-percolate and the flow-through/F4 were tested as they appeared in their original concentrations right off the columns, only diluted 1:20 and 1:100 as described herein. The culture of peripheral blood mononuclear cells (“PBMC”) is prepared with two vials of frozen PBMCs that were obtained from normal healthy human subjects by a commercial vendor and were added to 2×10 ml medium and centrifuged. PBMCs were resuspended and cultured in RPMI1640/5% FBS for 24 h. (1 vial of frozen cells in 11 ml medium).

Treatment agents for this exemplary method comprise three agents: unseparated phyto-percolate (‘PAZ’), fraction 3 (‘F3’), fraction 4 (‘F4’). Treatment concentration for each was 1:20 & 1:100. An exemplary sample preparation method for each agent by dilution is as follows: First, a 1:10 dilution is prepared by combining 0.7 ml agent (either PAZ, F3 or F4)+6.3 ml RPMI1640/5% FBS to obtain a total volume of 7 ml of a 1:10 solution. Second, a 1:50 dilution is prepared by combining 1.2 ml of the 1:10 dilution (of each respective agent)+4.8 ml RPMI1640/5% FBS for a total volume of 6 ml of 1:50 solution. In addition, for diluted fraction 3 (F3), 1M NaOH was used to adjust pH to 7.0.

According to this exemplary embodiment, seeding, treatment, and detection is accomplished by the following steps. Two dishes of PBMCs were combined and the small amount of PBMCs was stained with 0.4% Trypan blue and the cell number of PBMCs was counted using known techniques.

In this embodiment, an enzyme linked immunosorbent assay (“ELISA”) analysis of inflammatory cytokine secretion, a protocol provided in a commercial kit for the parallel quantification of the production of human cytokines was employed. The PBMC were first seeded into a twenty-four well plate (337,600 cells/each well in 3200 medium) and incubated at 37° C. for forty-eight hours. In this exemplary embodiment, an additional 320 μl of culture medium was added, and cells were cultured for 48 hours. For the control cultures, the 320 μl of additional medium contained no additional components. To stimulate the production of several cytokines, parallel cultures of PBMC were treated with 50 ng/ml phorbol myristate acetate (‘PMA’) and 1 μg/ml ionomycin for 24 hr, followed by addition of 0.64 μl PMA/0.64 μl ionomycin and incubation for an additional twenty-four hours. For cultures in which PBMC were treated with phyto-percolate or fractions derived therefrom, the 320 μl of additional medium which contained 1:10 or 1:50 dilutions of phyto-percolate or fractions 3 or 4 derived therefrom (to yield final dilutions in the cultures of 1:20 or 1:100) was added just before incubation for 24 hr, and then incubated with or without PMA+ionomycin treatment for an additional 24 hours. Duplicate PBMC cultures were examined for each of these conditions. At the end of the incubation period, the cultures were centrifuged and the supernatant medium was collected and aliquots stored at −70° C. The quantity of cytokines present in each of the culture medium samples was subsequently determined using a Multi-Analyte ELISArray Kit (product number MEH-004A) for human inflammatory cytokines and methods provided by SA Biosciences.

Analysis of the effect of phyto-percolate or fractions isolated therefrom on the DNA-binding activity of NF-κB in the nuclear protein fractions of the cultured PBMC was determined as follows in this exemplary embodiment: approximately 18.26 mL of suspended PBMC were added to approximately 18 mL of culture medium and 2 mL of this cell suspension (2,718,000 cells in 2 mL) were seeded into each 60 mm culture dish. In this exemplary embodiment, an additional 2 mL of culture medium was added. For the control cultures, the 2 mL of additional medium contained no additional components. Culture of cells stimulated with TNF-α was performed identically, including addition of 2 mL of additional medium at the start of the culture, but 2 μL of TNF-α (50 ng/ml) was added to these cultures one hour before harvesting. For cultures in which PBMC were treated with phyto-percolate or fractions derived therefrom, the 2 mL of additional medium contained 1:10 or 1:50 dilutions of phyto-percolate or fractions 3 or 4 derived therefrom (to yield final dilutions in the cultures of 1:20 or 1:100) was added just before incubation. Two positive controls for the inhibition of the DNA binding activity of NF-κB were performed. In one case, PBMC were cultured for 24 h in the presence of 25 μM G2535 for 24 h followed by TNF-α treatment for 1 h. In the second case, PBMC were cultured for 24 h in the presence of 25 μM Genistein for 24 h followed by TNF-α treatment for 1 h. Duplicate PBMC cultures were examined for each of these conditions which were then cultured at 37° C. for 24 hr before harvesting.

At the end of the incubation period, nuclear proteins were extracted from the cells according to the method of set forth in PubMed—Cancer Research 65:6934, 2005 and electrophoretic mobility shift assays (“EMSA”) were performed for the binding of NF-κB to a synthetic radiolabeled DNA sequence corresponding to the cognate NF-κB DNA-binding element using an established protocol such as the one set forth in PubMed—Cancer Research 65:6934, 2005.

Turning now to FIGS. 1-8, the methods of effecting various cytokines and NF-κB with the phyto-percolate, which is denoted by the phrase “PAZ”, and fractions thereof are discussed according to certain exemplary embodiments of the present invention. Although specific examples of the composition effecting the production of various cytokines and the DNA-binding activity of NF-κB are discussed herein, the present invention is not limited to only those examples or the compositions and quantities, dilutions, or fractions of the compositions discussed herein although Applicants reserve the right to claim certain quantities, dilutions, or fractions at a later date.

With specific reference to FIGS. 1A-1D, raw data is shown from various electrophoretic mobility shift assays or (“EMSA”) for NF-κB performed using a deoxyoligonucleotide corresponding to the DNA sequence to which NF-κB binds, labeled with an infrared dye. Specifically, FIGS. 1A and 1B depict both results in “low density” in which the bands were visualized using an infrared scanner (Li-Cor Corporation) for a short period of time (FIG. 1A) and in “high density” in which image obtained from the same gel was enhanced (FIG. 1B). FIGS. 1C and 1D depict the results when the tests resulting in the assays shown in FIGS. 1A and 1B were re-run for a longer time period (three hours compared to two hours) using an identical amount of the nuclear protein.

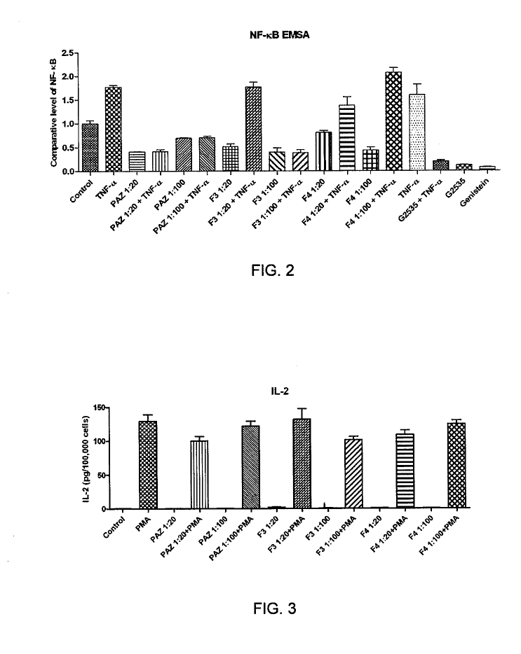

Turning now to FIG. 2, the effects of administering phyto-percolate, as well as various fractions that were obtained by chromatographic treatment of the complete phyto-percolate composition, on the DNA-binding activity of NF-κB in PBMC with or without stimulation with phrobol myristate acetate (PMA) are shown according to certain exemplary embodiments of the present invention. Active NF-κB is a dimeric protein that binds to a cognate DNA sequence to control the transcription of specific proteins that play key roles in inflammation. Therefore, the more NF-κB that is expressed and that binds to DNA, the greater the amount of inflammatory proteins that will be produced and the greater the inflammatory response. Reducing the overall amount of NF-κB that binds to DNA sequence of NF-κB target genes lowers the inflammation as well as reduces the other effects of NF-κB such as reducing the activation of various viruses such as the HIV virus.

As shown in FIG. 2, control, unstimulated and untreated PBMC were tested to determine the native amount of NF-κB that binds to a radiolabeled DNA probe. This represents a baseline measurement of NF-κB activity that is expressed as a relative unit of 1.0. According to this example, when tumor necrosis factor alpha or TNF-α was added, the DNA-binding activity of NF-κB was significantly increased to a relative level of almost 2.0. However, when a composition comprised of 1:20 dilution of phyto-percolate (labeled ‘PAZ’) was added to the PBMC, the concentration of NF-κB decreased significantly compared to the control to a relative level of approximately 0.4 units. As depicted in FIG. 2, and according to various exemplary embodiments of the present invention, phyto-percolate in a 1:20 and 1:100 dilution when combined with TNF-α, phyto-percolate in a 1:100 dilution alone, fractions 3 and 4 (labeled “F3” and “F4”) alone in a 1:20 and 1:100 dilution, and fraction 3 in a 1:100 dilution in the presence of TNF-α, reduced the overall concentration of NF-κB compared to the control, whereas fraction 4 in 1:100 dilutions plus TNF-α increased NF-κB concentration. FIG. 2 also depicts the results of adding TNF-α, G2535 plus TNF-α, G2535 alone, and genistein alone. As shown in FIG. 2, phyto-percolate alone, fraction 3 and fraction 4 inhibited NF-κB and both phyto-percolate and fraction 3 inhibited TNF-α induced activation of NF-κB.

Therefore, administering phyto-percolate may decrease the DNA-binding activity of NF-κB which in turn reduces inflammation. Further, since NF-κB activation promotes the replication and/or function of certain viruses, such as the HIV virus, reducing the total DNA-binding activity of NF-κB may reduce or prevent the pathological effects of certain viruses, such as HIV. The present invention contemplates that any effects of reduced DNA-binding activity of NF-κB now known or discovered in the future can be achieved by administering an effective amount of phyto-percolate and dilutions, fractions and derivatives thereof.

Turning now to FIGS. 3-5 and in accordance with various exemplary embodiments of the present invention, the effect of phyto-percolate on the production by PBMC of various interleukins is discussed. While certain specific interleukins such as IL-10, and IL-17A are discussed, phyto-percolate also has effects on other interleukins and in other inflammatory pathways.

With particular reference to FIG. 3, the quantity of IL-2 produced (expressed as pg of IL-2/100,000 cells) was measured following the addition of phyto-percolate and various dilutions and fractions thereof to PBMC in the absence of other stimulants, or when added to PBMC treated with PMA, according to one exemplary embodiment of the present invention. As shown, a control consisting of untreated cultured PBMC did not secrete a detectable quantity of IL-2 into the culture medium, whereas additions of PMA to the cultured PBMC resulted in secretion of approximately 125 pg/100,000 cells IL-2. Treatment of cultured PBMC with a 1:20 or 1:100 dilution of phyto-percolate did not induce production of detectable quantities of IL-2 (i.e. approximately the same results as for control, untreated PBMC). The addition of a 1:20 dilution of phyto-percolate, fraction 3 in a 1:100 dilution and fraction 4 in a 1:20 dilution to PBMC stimulated with PMA reduced the production of IL-2 compared to PBMC treated with PMA alone. Treatment of cultured PBMC with fraction 3 and fraction 4, derived from chromatographic fractionation of phyto-percolate, at 1:20 and 1:100 dilutions did not induce production of detectable quantities of IL-2, similar to the control. However, according to this exemplary embodiment, when phyto-percolate in a 1:100 dilution and fraction 3 of phyto-percolate in a 1:20 dilution and fraction 4 of phyto-percolate a 1:100 were tested on PBMC in the presence of PMA, the overall amount of IL-2 did not change significantly when compared with the addition of PMA alone.

Therefore, as depicted in this exemplary embodiment, the addition of phyto-percolate and dilutions, fractions or derivatives thereof may reduce the concentration of IL-2 produced by PBMC in response to agents that stimulate IL-2 production, but they neither do not stimulate the production of IL-2 themselves, nor do they potentiate the production of IL-2 by agents known to induce production of this cytokine (for example PMA). The action of phyto-percolate to reduce (or not to increase) the production of IL-2 by PBMC reflects its ability to reduce the amount of inflammation as well as other effects of IL-2 now known or discovered in the future. According to various exemplary embodiments of the present invention, the ability to not up-regulate an inflammatory cytokine such as IL-2 while simultaneously up-regulating an anti-inflammatory cytokine such as IL-10 is effective at reducing the amount of inflammation and is superior to conventionally available therapies as it reduces undesirable side effects.

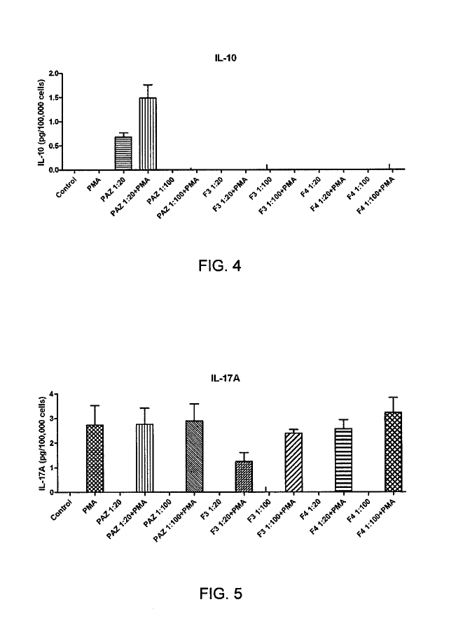

Turning now to FIG. 4 and in accordance with yet another exemplary embodiment of the present invention, FIG. 4 depicts the overall production and secretion of IL-10 (expressed as pg of IL-10/100,000 cells) when phyto-percolate, various fractions and dilutions thereof, and PMA are added to cultured PBMC. As shown in FIG. 4, the phyto-percolate in a 1:20 dilution alone and in a 1:20 dilution tested in conjunction with PMA increased the overall secretion of IL-10 compared to control PBMC, which did not secrete detectable quantities of IL-10 into the medium. In this one exemplary embodiment as shown, the various other dilutions and fractions of phyto-percolate alone or in combination with PMA did not appear to effect the overall concentration of IL-10. However, as in the cases with the other exemplary embodiments depicted herein, fractions 3 and 4 comprise only a small percentage of the composition of phyto-percolate and this result does not limit the invention to the point where phyto-percolate in the concentrations and fractions that did not increase IL-10 concentration necessarily cannot ever increase IL-10 concentration.

Therefore, phyto-percolate may increase the overall concentration of IL-10. Increasing the overall concentration of IL-10 should reduce the amount of inflammation as IL-10 is an anti-inflammatory cytokine. Further, the present invention contemplates that the other effects now known or discovered in the future that are attributable to IL-10 can be achieved by the addition of phyto-percolate.

According to various exemplary embodiments of the present invention, phyto-percolate's effects to reduce inflammation can occur due to its effect of reducing the DNA-binding activity of NF-κB, alone or in combination with increasing the production and secretion of anti-inflammatory cytokines such as IL-10 and by reducing inflammatory cytokines such as IL-2 or tumor necrosis factor-alpha (“TNF-α”) as noted below. Therefore, the present invention contemplates that phyto-percolate has effects on multiple different cytokines at one time to achieve an overall effect, such as reducing inflammation according to various exemplary embodiments.

With reference now to FIG. 5, and in accordance with one exemplary embodiment of the present invention, the addition of phyto-percolate to a mixture of cultured PBMC to effect the overall production and secretion of IL-17A (expressed as pg of IL-17 secreted/100,000 cells) is disclosed. Besides IL-17A, interleukin 17 (synonymous with interleukin 17A) is similarly affected by the addition of phyto-percolate. As shown, unstimulated cultured control PBMC do not secrete detectable levels of IL-17A whereas the addition of PMA to cultured PBMC resulted in a significant increase of IL-17A to approximately 3 pg/100,000 cells. The addition of phyto-percolate in a 1:20 dilution or a 1:100 dilution did not result in detectable secretion of IL-17A from control PBMC, and the addition of 1:20 dilution or a 1:100 dilution of phyto-percolate or fraction 4 in a 1:100 dilution in the presence of PMA did not cause any change in the levels of IL-17A secreted in response to PMA alone. Fraction 3 and fraction 4 of phyto-percolate in both 1:20 dilution and 1:100 dilution did not result in detectable secretion of IL-17A from control PBMC. An addition of fraction 3 of phyto-percolate in a 1:20 dilution significantly reduced the secretion of IL-17A by PBMC in response to PMA treatment to approximately 1 pg/100,000 cells. Fraction 3 of phyto-percolate in a 1:100 dilution as well as fraction 4 of phyto-percolate in a 1:20 dilution also reduced the section of IL-17A by PBMC in response to PMA treatment as shown.

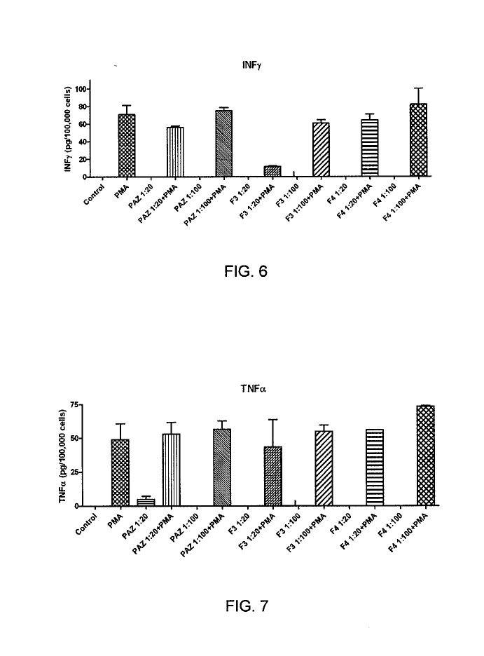

With reference to FIGS. 6-8, the effect of phyto-percolate on other cytokines is disclosed. Specifically, the effect of phyto-percolate in various dilutions and fractions on interferon-gamma (IFN-γ), tumor necrosis factor-alpha (TNF-α), and granulocyte macrophage colony stimulating factor (GM-CSF) is disclosed.

As shown in FIG. 6, and in accordance with one exemplary embodiment of the present invention, the effect of phyto-percolate on the concentration of IFN-γ (expressed as pg of IFN-γ secreted/100,000 cells) is disclosed. According to this exemplary embodiment, unstimulated cultured control PBMC do not secrete detectable levels of IFN-γ whereas the addition of PMA to cultured PBMC resulted in significant secretion of IFN-γ to approximately 70 pg/100,000 cells. While the addition of phyto-percolate to cultured PBMC in a dilution of 1:20, a dilution of 1:100, or fraction 3 or fraction 4 in these dilutions did not result in the secretion of detectable levels of IFN-γ in this exemplary embodiment, the addition of phyto-percolate in a dilutions of 1:20 to PBMC in combination with PMA decreased the overall secretion of IFN-γ that is induced by PMA alone. The addition of fraction 3 of phyto-percolate in a 1:20 dilution significantly decreased the PMA-induced secretion of IFN-γ to approximately 10 pg. Fraction 3 of phyto-percolate in a 1:100 dilution decreased the PMA-induced secretion of IFN-γ to approximately 60 pg as did fraction 4 of phyto-percolate in a 1:20 dilution.

Therefore, phyto-percolate does not induce the production of IFN-γ and may modulate the overall production of IFN-γ caused by other agents and thus enable the benefits that may be derived therefrom.

With reference now to FIG. 7 and in accordance with an exemplary embodiment of the present invention, effect of phyto-percolate on the production and secretion of TNF-α (expressed as pg secreted/100,000 cells) was measured. According to this exemplary embodiment, unstimulated cultured control PBMC do not secrete detectable levels of TNF-α whereas the addition of PMA to cultured PBMC resulted in significant secretion of TNF-α to approximately 50 pg/100,000 cells. The phyto-percolate in a 1:100 dilution or fraction 3 in a 1:20 or 1:100 dilution or fraction 4 of phyto-percolate in a 1:20 or 1:100 dilution do not induce the secretion of detectable levels of TNF-α. Phyto-percolate and fractions derived therefrom did not significantly alter the PMA-induced secretion of TNF-α by cultured PBMC.

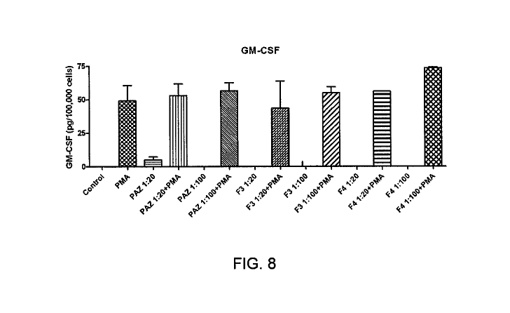

Turning now to FIG. 8 and in accordance with another exemplary embodiment of the present invention, the effect of administering various concentrations and fractions of phyto-percolate on the production and secretion of GM-CSF by PBMC (expressed as pg secreted/100,000 cells) is discussed. As shown, a control consisting of unstimulated cultured PBMC did not produce a measurable amount of GM-CSF, whereas the addition of PMA induced the secretion of approximately 50 pg/100,000 cells. Phyto-percolate in a 1:20 dilution induced the secretion of a very low level GM-CSF (approximately 5 pg/100,000 cells) whereas a 1:00 dilution of phyto-percolate or various dilutions of fractions 3 and 4 did not induce GMCSF secretion. Further, phyto-percolate as well as fraction 3 in both a 1:20 dilution and a 1:100 dilutions and fraction 4 at 1:20 dilution did not influence the production of GM-CSF by PBMC in the presence of PMA.

Therefore, according to these exemplary embodiments, phyto-percolate by itself in various dilutions and fractions does not cause the secretion of appreciable quantities of GM-CSF and phyto-percolate in various dilutions and fractions does not significantly alter the production of GM-CSF that is induced as the result of treatment by other agents.

Therefore, according to various exemplary embodiments of the present invention, the administration of phyto-percolate regulates various cytokines and NF-κB to achieve certain desired effects such as the reduction of inflammation. Unlike compositions of the prior art, phyto-percolate can regulate multiple cytokines to achieve reduced inflammation. For example, as shown and discussed above, the administration of phyto-percolate can up-regulate IL-10 without up-regulating IL-2 to greater reduce inflammation.

Further, phyto-percolate and various dilutions and fractions thereof are capable of inhibiting NF-κB and TNF-α induced activation of NF-κB thus indicating that phyto-percolate functions as an antioxidant. Also, according to certain exemplary embodiments, administering phyto-percolate in various dilutions and fractions, especially fraction 3, significantly inhibits the DNA-binding activity of NF-κB. Administering an effective amount of phyto-percolate will not induce certain pro-inflammatory cytokines such as TNF-α or IFN-γ, while inducing various anti-inflammatory cytokines such as IL-10, to reduce inflammation. Further, according to these various exemplary embodiments, the administration of phyto-percolate did not have a toxic or irritant effect on cells or tissue.

It should be understood that various principles of the invention have been described in illustrative embodiments. However, many combinations and modifications of the above-described formulation, proportions, elements, materials, and components used in the practice of the invention, in addition to those not specifically described, may be varied and particularly adapted to specific environments and operating requirements without departing from those principles. Other variations and modifications of the present invention will be apparent to those of ordinary skill in the art, and it is the intent that such variations and modifications be covered by this disclosure.

What is claimed is:

1. A method of treating inflammation; the method comprising administering an effective amount of phyto-percolate derived from culturing microorganisms of ATCC Deposit #PTA-5863 wherein the inflammation is treated by the phyto-percolate upregulating anti-inflammatory cytokines while down regulating pro-inflammatory cytokines.

2. The method of claim 1, wherein the anti-inflammatory cytokines comprise IL-10 and the inflammatory cytokines comprise IL-2.

3. The method of claim 1, wherein the inflammatory cytokines comprise TNF-α.

4. The method of claim 1, wherein the inflammatory cytokines comprise IFN-γ.

5. The method of claim 1, wherein the anti-inflammatory cytokines comprise IL-10 and the inflammatory cytokines comprise IL-2, TNF-α, and IFN-γ.

6. The method of claim 1, wherein the phyto-percolate affects the expression of cytokines on a cellular level.

7. The method of claim 1, wherein the phyto-percolate further affects the activation of NF-κB.

8. A method of affecting NF-κB; said method comprising administering an effective amount of phyto-percolate derived from culturing microorganisms of ATCC Deposit #PTA-5863.

9. The method of claim 8, wherein the affecting of NF-κB occurs by decreasing expression, activation or the DNA-binding activity of NF-κB.

10. The method of claim 8, wherein the affecting of NF-κB reduces inflammation.

11. The method of claim 8, wherein the affecting of NF-κB results in affecting various viruses.

12. The method of claim 11, wherein the viruses comprise the HIV virus.

13. The method of claim 8, wherein the affecting of NF-κB treats or prevents disorders of the immune system, including cancer.

14. The method of claim 8, wherein the affecting of NFκB affects host immune response.

15. A method of affecting cytokines; said method comprising administering an effective amount of phyto-percolate derived from culturing microorganisms of ATCC Deposit #PTA-5863.

16. The method of claim 15, wherein the cytokines affected comprise interleukins.

17. The method of claim 15, wherein the cytokines affected comprise TNF-α.

18. The method of claim 15, wherein the cytokines affected comprise IL-2, IL-10, IL-17A, and IL-17 and the affecting of the IL-2, IL-10, IL-17A, and IL-17 results in the regulation of the immune response.
